(19) United States
(12) Patent Application Publication
(10) Pub. No.: US 2008/0156319 A1
(43) Pub. Date: Jul. 3, 2008

(54) Pulsating Inhaler and a Method of Treating Upper Respiratory Disorders

(76) Inventor: Yuval Avni, Giv'ataim (IL)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 11/916,306

(22) PCT Filed: May 24, 2006
(86) PCT No.: PCT/IL06/00611

§ 371 Date: Dec. 3, 2007

(30) Foreign Application Priority Data
Jun. 2, 2005 (IL) 168975

(51) Int. Cl. A61M 11/00; A61M 15/00; A61M 16/00

(52) U.S. Cl. 128/200.14; 128/200.21; 128/200.22; 128/203.15; 128/204.18

(57) ABSTRACT

The present invention discloses a pulsating inhaler, comprising a fluid oscillator providing a focused fluid column with a series of alternating high and low pressures zones; a medicament dispenser adapted for releasing small and constant measures of at least one medicament via said fluid column; and, at least one outlet orifice adapted to direct said focused fluid column towards the respiratory tract of a patient, wherein said small and constant measures of medicament are subjected to the patients while its respiratory tracts are gently and continuously vibrated The present invention also discloses a method of inhaling small and constant measures of medicament while gently and continuously vibrating the patient's respiratory tracks by a means of a fluid column characterized by a series of alternating high and low pressures zones.

### FIELD OF THE INVENTION

The present invention generally relates to a pulsating inhaler, and to a method of treating the upper respiratory disorders.

### BACKGROUND OF THE INVENTION

Upper respiratory disorders such as viral upper respiratory tract infections or “common cold”, allergic rhinitis, and rhinosinusitis are associated with impairment in mucociliary clearance in the nasal passages. Although the causes of these disorders are varied, they share a common set of nasal symptoms such as rhinorrhea, nasal congestion/blockage, and post-nasal drip. In these conditions the mucous membranes of the nose and paranasal sinuses become irritated, leading to symptoms. In some patients, this irritation is sufficient to hinder the normal drainage of the sinuses into the nasal cavity, resulting in blockage that may lead to additional impaired ciliary activity, intense pressure/pain, and increased likelihood of infection.

Allergic rhinitis (AR) is a condition that results from exposure to allergens, either at specific times of the year (seasonal allergic rhinitis) or year-round (perennial allergic rhinitis). Up to one-half of AR patients suffer from both seasonal as well as perennial AR, approximately one-third suffer from seasonal AR alone and another one-third from perennial AR alone. In either seasonal or perennial AR, the symptoms and treatment approaches are similar. Symptoms most often include nasal congestion or stuffiness, rhinorrhea and nasal itching. Allergic rhinitis affects nearly 150 million people in the world's seven major pharmaceutical markets, and annual sales of prescription products to treat allergic rhinitis are estimated to total more than $4.5 billion worldwide. The treatments currently available include primarily prescription and over-the-counter antihistamines, decongestants and nasal corticosteroids, delivered by nasal sprays, evaporation devices, and ointments.

Upper Respiratory Tract Infections (URTI) and the common cold affect all ages and are uncomfortable conditions with lost work and school days.

Asthma and COPD (chronic obstructive pulmonary disease) are chronic illnesses requiring lifetime therapy and affect 44 million Americans. The mainstay of treatment is inhaler therapies. The delivery of medications via inhaler is problematic causing decreased efficacy and poor patient compliance. Therefore there is a constant search for improving the delivery of drugs through inhalers.

The problem with current inhalers is the tendency of deposit of the medication in the oral cavity, not in the lungs where it is effective. Also, it is difficult to coordinate the delivery of the drug with the expiratory cycle. Because of these problems the accurate dosage of medication cannot be delivered and the treatment causes many side effects. In addition the current inhaler techniques are passive techniques that are based on the ability of the patient to suck the drug to the lungs. As their action on the patient is solely and totally drug-dependent, the above-mentioned faults cause a major problem to the user.

The pulsating inhaler delivery system works on the principal of successive small pulses of air (that can contain powder or liquid) in metered doses. This enhances delivery directly to lungs and increase efficacy. It does not require positioning or coordination with breathing. Therefore, there is minimal deposition in the oral cavity and minimal associated morbidity, e.g., dry mouth, bad taste, fungal infections. By delivering the medication with pulsation of small doses there will be an increase in bioavailability thus increasing the effectiveness of current inhalation medications. This method will be suitable for all medications, therefore very attractive to the patients. There will be an increase in patient compliance due to ease of use and increased feeling of effectiveness without the side effects that are currently a problem.

The pulsating inhaler will deliver drugs using unique device specific cartridges that will be disposable.

The efficient delivery of drugs to the lungs can also serve as a treatment modality for various systemic diseases such as the delivery of inhaled insulin to treat diabetes patients. These applications require accurate dosage that is difficult to achieve with the current inhaler technology.

The device has also non-drug dependent physiologic functions as it transfers vibration to the airways. Thus causing smooth muscle relaxation, reduction of airway edema, increase in blood and lymphatic flow, improved gas exchange and oxygenation and decreased dyspnea.

As the device continue to oscillate during inspiration and expiration it provides a pulsating continuous positive airway pressure (PCPAP). The PCPAP prevents the collapse of the airways of the asthma and COPD patient, during expiration, which decreases dyspnea and is highly beneficial to the patients.

### BRIEF DESCRIPTION OF THE FIGURES

In order to understand the invention and to see how it may be implemented in practice, a preferred embodiment will now be described, by way of non-limiting example only, with reference to the accompanying drawing, in which

FIG. 1 schematically presenting a lateral section of a pistol pulsed inhaler according to one simple embodiment of the present invention;

FIGS. 2a and 2b schematically presenting a lateral cross section (upper view) of a piston pulsed inhaler according to yet another embodiment of the present invention;

FIGS. 3a and 3b schematically presenting a lateral cross section of a disc pulsed inhaler according to yet another embodiment of the present invention; and,

FIGS. 4a-4c schematically presenting a plurality of lateral cross sections of a disc pulsed inhalers according to the same embodiment of the present invention; and,

FIGS. 5a and 5b schematically presenting a lateral cross-section of a disc-inhaler according to yet another embodiment of the present invention; said device comprising at least one valve adapted to facilitates medicament dispensing at both steps of inhalation (illustration on the right) and exhalation (illustration on the left).

### SUMMARY OF THE INVENTION

It is thus one object of the present invention to provide a cost effective and highly useful pulsed inhaler. This light and portable device is inter alia comprised of a fluid oscillator providing a focused fluid column (e.g., to a dispersed cloud or aerosol of a medicament generally directed towards the patient head) with a series of alternating high and low pressures zones; a medicament dispenser adapted for releasing small and constant measures of at least one medicament via said fluid column; and at least one outlet orifice adapted to direct said focused fluid column towards the respiratory tract of a patient. The small and constant measures of medicament are subjected to the patients while its respiratory tracts are gently and continuously vibrated.

Still another object of the present invention is to provide a useful method of inhaling small and constant measures of medicament while gently and continuously vibrating the patient's respiratory tracts by a means of a focused fluid column characterized by a series of alternating high and low pressures zones.

A last object of the present invention is to provide a useful method of preventing airways collapse during expiration in asthma and COPD patient, providing an accurate and efficient systemic drug delivery system through inhalation diseases of the upper respiration tract and diseases of the ears. This method is thus utilizable for treating respiratory diseases, and especially useful for reducing dyspnea, edema, spasm and collapse of the airways. The method comprising vibrating the inhaled and/or exhaled air column while dispensing accurate and small dosage of medicaments to the respiratory tracks and peripheral regions wherein reducing the deposition of said medicament into the mouth cavity. The method is especially useful for preventing airways collapse during expiration by providing PCPAP and treating rhinitis, allergic rhinitis, rhinosinusitis, URTI, asthma and/or COPD.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a pulse inhaler and method of pulsed inhaling.

The term ‘inhaler’ refers hereinafter to an apparatus used for inhaling medicinal substances or air. The inhaler according to the present invention comprising a fluid oscillator providing a focused fluid column with a series of alternating high and low pressures zones; a medicament dispenser adapted for releasing small and constant measures of medicaments via said fluid column; and at least one outlet orifice adapted to direct said focused fluid column towards the respiratory tract of a patient, wherein said small and constant measures of medicaments are subjected to the patients while its respiratory tracts are gently and continuously vibrated.

According to one embodiment of the present invention, the pulsed fluid is a comprisable gas, such as air, carbon dioxide etc. Additionally or alternatively, the fluid is in a liquid form, such as water or other water miscible solution, organic solvents, singled-phased or multi-phased fluids, homogeneous or heterogeneous diluents or any combination thereof. A mixture of liquid and gas and fine particles containing fluids are also possible.

According to one embodiment of the present invention, the inhaler additionally comprises a heating/cooling means, wherein the temperature of the fluid is conditioned to a predetermined measure before being subjected to the respiratory tracts of the patient. Electrical heating means adapted to elevate the temperature of the inhaled fluid from its ambient temperature to about 37° C. to 42° C. is preferred. Humidifying means are also possible to be incorporated.

According to one embodiment of the present invention, the medicament is in solid phase. Hence, the medicament may be selected from granular matter, a drug sized to form fine particles, powder, sol, gel, sol-gel, glass, encapsulated matter, milled composition or any combination thereof. Alternatively or additionally, the medicament may be utilized in a liquid phase. In such a case, the fluid is selected in a non limiting manner from water miscible compositions, water immiscible compositions, emulsions, extracts, dispersions, suspensions, vasiculated solutions, aggregated phases or any combination thereof.

It is according to another embodiment of the present invention wherein the fluid or medicament is selected in a non-limiting manner from at least one of the group of Braochodilators, especially sympatic mimetics, alfa antagonists, anti cholinergics; nasal decongestants, such as pseudoehedrines, ephedrines; steroids; anti histamines; anti prostaglandins, alternative or homeopathic medicaments; vaso constrictors; local anesthetics; mast cell stabilizers; antibiotics, such as biocides, fungicides etc; pleasant odor; pheromones; hormone treatments, such as ADH, insulin, growth hormones; vapors, humidifiers; drying compositions; hot or cold vapors; hyper-, iso- or hypotonic vapors or any combination thereof, or decongestants, essential oils, volatile compounds, etheric oils, terepenes, terpanols and either water miscible or water-immiscible extracts, especially oils or extracts obtained from Amyris, Balsam, Bay Rum, Black Pepper, bornyl acetate, Cajeput, Camphor, Cedarwood, Cedarleaf oil, Chamomile, chlorbutanol, Cinnamon, Clary Sage Rosewood, Clove, Eucalyptus, Frankincense, Geranium, Ginger, Lavender, Lemon, Lemon essential oils, levomenthol, Lime, Menthol, Mint, Myrrh, nutmeg oil Orange, Patchouli, Peppermint, Pine Needle, Rose Eucalyptus, rosemary, Rosewood, Sage, Sandalwood, Spearmint, Tea Tree, terpinol, turpentine oil, thymol, Ylang Ylang or any combination thereof.

Otherwise, and still in a non-limiting manner, the aforesaid medicament is at least one of the group of compositions and commercial available medicaments, their derivatives, or by-products provided thereof, selected from: Acrivastine, Aller-Eze Clemastine, Aller-Eze nasal spray, Azatadine maleate, Azelastine nasal spray, Beclometasone nasal spray, Beclometasone nasal spray, Beconase hayfever nasal spray, Beconase hayfever relief for adults, Beconase nasal spray, Benadryl allergy relief, Benadryl, Benadryl, Benadryl plus, Brompheniramine maleate, Budesonide nasal spray, Calimal Antihistamine, Cetirizine, Chlorphenamine, Clarityn, Clemastine, Cyproheptadine hydrochloride, Desloratadine, Dexa-Rhinaspray Duo. Dimotane elixir, Dimotane plus, Dimotapp elixir, Dimotapp elixir paediatric. Dimotapp LA, Flixonase allergy nasal spray, Flixonase aqueous nasal spray, Fluticasone propionate nasal spray, Galpharm hayfever and allergy relief, Galpseud Plus, Haymine, Histafen, Ipratropium bromide nasal spray, Levocabastine nasal spray, Levocetirizine dihydrochloride, Livostin direct nasal spray, Livostin nasal spray, Loratadine, Medised, Medised, Mistamine, Mizolastine, Mizollen, Mometasone furoate nasal spray, Nasacort, Nasobec nasal spray, Nasonex nasal spray, Neoclarityn tablets/syrup, Optimine syrup, Periactin, Phenergan, Piriteze, Piriton, Pollenase hayfever nasal spray, Promethazine hydrochloride elixir, Promethazine hydrochloride, Rhinocort Aqua, Rhinolast allergy nasal spray, Rhinolast nasal spray, Rinatec nasal spray, Rino clenil nasal spray, Rynacrom allergy nasal spray, Rynacrom nasal spray, Semprex, Sodium cromoglicate nasal spray, Sudafed Plus, Syntaris nasal spray, Tavegil, Telfast 120, Terfenadine, Terfinax, Triamcinolone acetonide, Vista-Methasone, Xyzal tablets, Zirtek allergy relief tablets, Zirtek allergy tablets/solution, Afrazine, Anadin, Beechams all-in-one, Beechams products, Benylin products, Contac, Day Nurse, Dimotapp elixir, Dimotapp elixir paediatric, Dimotapp products, Galpseud, Karvol decongestant products, Lemsip products, Meggezones, Merocets Plus lozenges, Nurofen Cold and Flu, Otrivine Menthol Nasal Spray, Otrivine Metered Dose Sinusitis Spray, Otrivine prducts, Pseudoephedrine hydrochloride, Sterwin real lemon cold powders and other products, Strepsils Menthol and Eucalyptus and other products, Sudafed and its products, Xylometazoline nasal drops, Bactroban Nasal, Fusafungine, Locabiotal, Naseptin nasal cream, Ipratropium bromide nasal spray, Rinatec nasal spray, pseudoephedrine, propylhexedrine, L-Desoxyephedrine, xylometazoline hydrochloride or any combination thereof. Inhaling of a dispersed medicament while vibrating the respiratory tracks thus is the core of the present invention.

It is according to another embodiment of the present invention wherein the medicament is being activated before or while being carried by the fluid column. Said activation is provided by physical, chemical and/or biological means. Hence for example, at least one ingredient of the medicament of at least a portion of the same is heated, radiated, emitted by UV, sonicated, vibrated etc; oxidized, polymerized, transesterified, hydrolyzed or subjected to enzymological reactions.

It is according to another embodiment of the present invention wherein the medicament is introduced to the dispenser by means of a disposable capsule or the like. Said capsule comprises an envelope adapted to be punctured such that at least a limited measure of the medicament contained in said enveloped is forced to flow toward the pulsed column of fluid in a predetermined flux.

It is according to another embodiment of the present invention wherein the fluid oscillator is at least one rotating pistol. Preferably, the pistol is actuated by means of a motor interconnected to an accentor. In this mechanism, an electrical or mechanic rotating motor is periodically actuating a shaft interconnected to said accentor and said pistol. The pistol compresses the fluid inside a pressure cabinet and segments the air column. Said fluid escapes from the pressure cabinet in predetermine intervals via at least one outlet orifice, adapted to direct and focus the fluid column towards the respiratory tracts of the patient. It is acknowledged in this respect that the fluid column is provided in an oscillated manner so as it composed of a series of ever changed lower and higher pressure peaks. This pulsed fluid column is especially adapted to vibrate the respiratory tracks of the patient. Thus causing smooth muscle relaxation, reduction of airway edema, increase in blood and lymphatic flow, improved gas exchange and oxygenation and decreased dyspnea.

The high pressure peaks of the fluid column carry the medicament to the patient's airways. Being carried in the high pressure peaks, the delivery of the drug is not relied on the patient's ability to suck the drug into his lungs and there are less drug deposits in the oral cavity and less side effects as a result.

While the interrupted fluid column gently and continuously vibrates the respiratory tracks of the patient, small portions of the medicaments are being carried through wider, more relaxed and less edematous airways further to the lungs and to the medium small sized airways, increasing efficacy and reducing the side effects to the patient. Said vibrations improve mucociliary clearance of secretions in lungs and airways, thus preventing accumulation of secretions and diseases.

Further so, the pulsation, when applied during expiration, can help to prevent airway collapse during asthma or COPD attack.

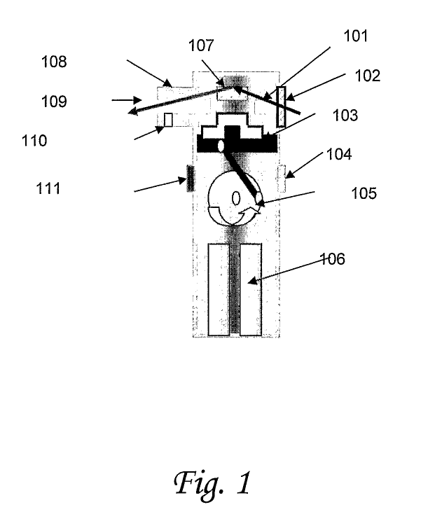

Reference is made now to FIG. 1, schematically presenting a lateral section of the inhaler according to one simple embodiment of the present invention. Said inhaler comprising a piston (103) actuate by a motor with an accentor (105). Air is forced to enter from adjustable inlet valve (102) (See arrow 101) to an optional heating chamber (107) and then (See arrow 109) said pulsed column of pressed air is forced outside via outlet orifice 108. Optionally, On/Off switch (104) is operating the device. One battery or more (106) supplies the electrical power. A plurality of rechargeable 1.5V batteries are presented in item number 108. Optional sensor 110 is also illustrated, adapted to determined the direction of air flow, in case air is possible either inhaled and/or exhaled through said inhaler.

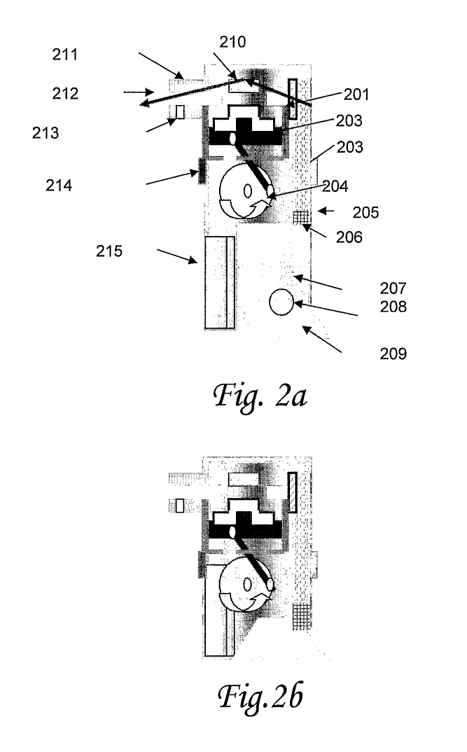

Reference is made now to FIGS. 2a and 2b, schematically presenting a lateral projection (upper view) of a pulsed inhaler according to yet another embodiment of the present invention. This medical device comprising turbine (201), piston (203), on/off switch (203), air inlet (205), dispensing module (206) adapted to batch or dispense disposable compositions and/or air, fluid container (207), fluid/medicament switching means (208), capsule housing (209), worm air stream (208) conditioned to about 37, dispensing rim (211) located adjacent to the respiratory tracks of the patient, and dispensing orifice (212) also denoted as fluid outlet, flow direction sensor (213), air condition switch (214), and couple of AC supply means (215).

Reference is still made to FIGS. 2a and 2b, lower view, presenting a similar inhaler configuration, wherein fluid container is omitted, and medicament is admixed with compressed air.

It is also in the scope of the invention, wherein the fluid oscillator is a rotating disc. The disc is of any shape or size. A disc missing a small section is especially preferred. Small portions of medicaments are dispersed towards the rotating disc, while a turbine continuously forces fluid (normally air) via a Bernulli hose, e.g., a conic pathway wherein the wide inlet sucks air which is forcefully streamed via a narrower outlet, such as constant measures of the medicament are ejected throughout and outlet orifice to the patient. The circulated disc and the turbine may share a single motor.

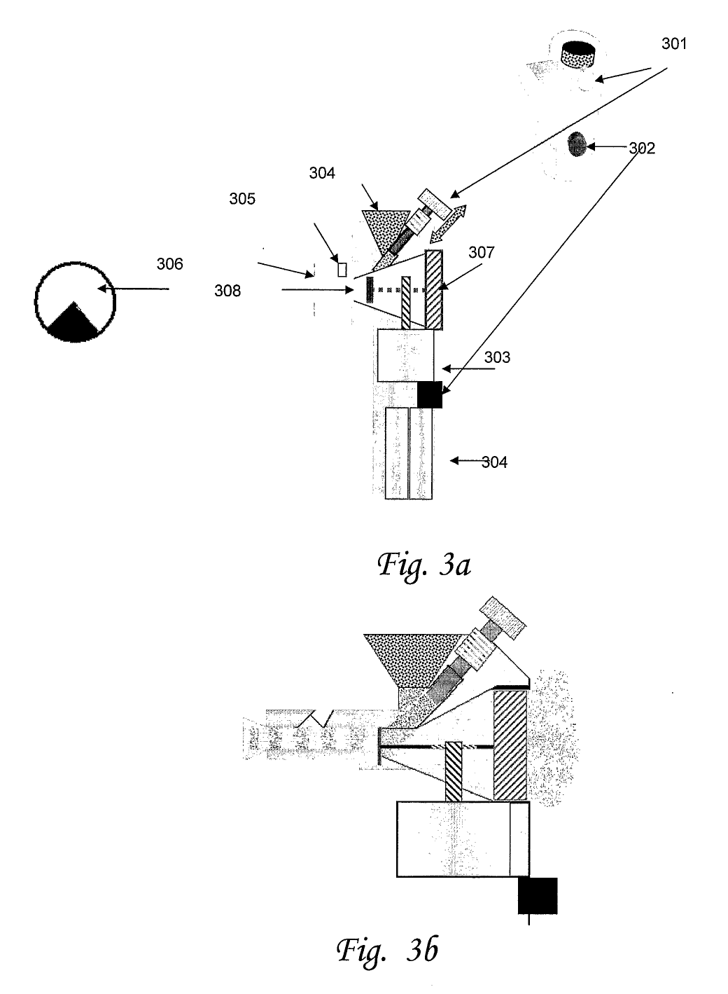

Reference is made now to FIGS. 3a and 3b, presenting a lateral section of a disc-containing pulse inhaler according to one embodiment of the present invention. Medicament dispensing pusher (301) is adapted to dispense small amount of medicament container in a medicament container (304). While a small batch of the medicament is dispensed, both an air turbine (307) and at least one rounded disc (307) with missing edge are spinned such that pulsed air is forced via outlet orifice (306). In this device, motor (303) actuates both turbine (307) and disc (308). An on/off switch (302) may regulate or activate the same. A top view of disc (308) is also presented (left side of FIG. 3). It is acknowledged in this respect that the amount of the dispersed medicament is also regulated by the size and shape of spinning disc (308) and its at least one missing edge. Optionally, a sensor (305) may be also provided. The device may contain a dosage indication window for the patient and an end-of life (end of doses) indicative window as well.

Lower view in FIGS. 3a and 3b schematically illustrates the disc-containing inhaler in a lateral section, showing the pulsed fluid column and the small medicaments portions carried by the same intervals.

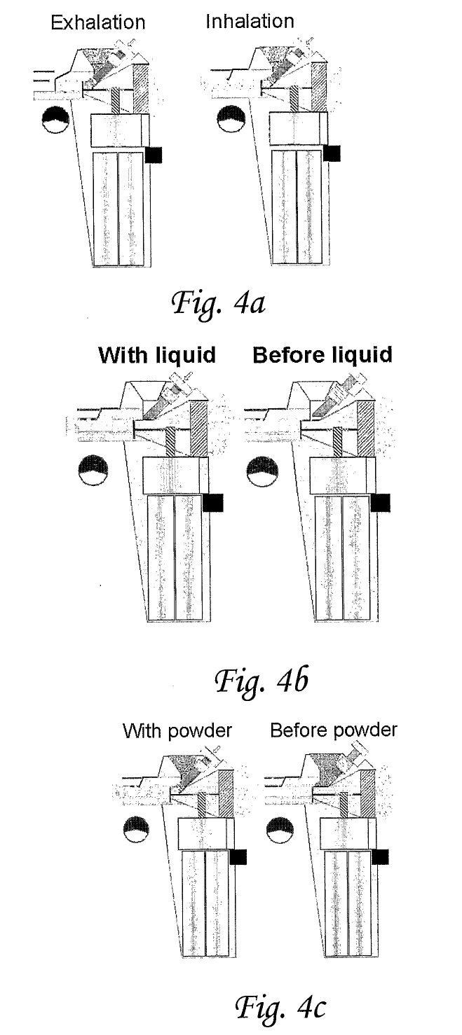

It is also in the scope of the present invention wherein the disc-containing inhalator as described in FIGS. 3a and 3b and 4a-4c is comprised of a fluid turbine (307); a medicament dispenser (304) adapted for releasing small and constant measures of at least one medicament via said fluid column; a fluid and medicament oscillator (308) providing a focused air or wind column with a series of alternating high and low medicaments concentration; said oscillator is one or more rotating discs, especially a discs comprising a missing edge; and, at least one outlet orifice (306) adapted to direct said focused fluid column towards the respiratory track of a patient, wherein said small and constant measures of medicament are subjected to the patients while its respiratory tracks are gently and continuously vibrated.

Reference is also made now to FIGS. 4a-4c, presenting a plurality of lateral sections of the same, operating in various stages of exhalation, inhalation, with powdered medicaments and before powder, with liquid medicaments and before said liquid dispensing.

According to a further embodiment of the present invention the medicament is contained into an envelope (capsule). The insertion of this capsule may initiate the medicament delivery by means of a micro-switch triggered by the detergent dispensing cabinet when said cabinet is closed. Said capsule may provides coded information for activating the dispensing of the medicament. It is another embodiment of the present invention in which the medicament delivery is provided by 2D or 3D specific and predetermined fitting mechanism, e.g., fitting by means of shape and sizes. In a further embodiment of the present invention the shape of the capsules inside the inhaler envelope provides key-in-lock information for activating the inhaler.

It is further in the scope of the present invention wherein the medicament is forced towards the client respiratory tracks directly as defined above or indirectly, e.g. via one or more dispensing means being either active or passive. Active dispensing means are selected in a non-limiting manner from medicament dispensers, such as inhalers of Ventolin™ (Salbutamol™) or the like, humidifiers etc. It is thus according to one embodiment of the present invention wherein the dispersed medicaments forced outside the invented device is fed into said active dispensers. Passive dispensing means are either flexible or rigid pipes, tubes and other conducting means adapted to force or purge at least a portion of the dispersed material towards a predetermined, e.g., focused target being either adjacent to the invented device or located in a remote location.

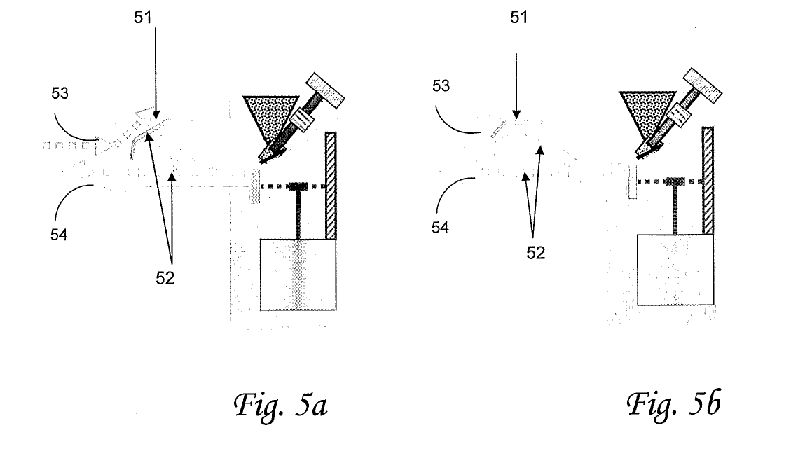

Reference is also made now to FIGS. 5a and 5b, presenting a valves-containing pulsed inhaler, here, a disc-inhaler. Said unidirectional valve or valves are adapted to separate between the inhalation and exhalation ever-changing steps or the respiratory mechanism. The figure on the left illustrates a cross-section of the inhaler in its closed-valves configuration at the exhalation step, wherein valve (51) is positioned in a manner that external air can not penetrate the inhaler via inhalation aperture (53) and dispensed medicament air column is exhaust via both exhalation aperture (54) and inhalation aperture (53). The figure on the right illustrates, however, a cross-section of same in its open-valves configuration at the inhalation step, wherein valve (51) is positioned in a manner that external air is directed to penetrate the inhaler via inhalation aperture (53), while dispensed medicament air column is exhaust solely via exhalation aperture (54).

1. A pulsating inhaler, comprising:
a. a fluid oscillator providing a focused fluid column with a series of alternating high and low pressures zones;
b. a medicament dispenser adapted for releasing small and constant measures of at least one medicament via said fluid column; and,
c. at least one outlet orifice adapted to direct said focused fluid column towards the respiratory tract of a patient,
wherein said small and constant measures of medicament are subjected to the patients while its respiratory tracts are gently and continuously vibrated.

2. The inhalator according to claim 1, wherein the fluid is a gas.

3. The inhalator according to claim 2, wherein the fluid is air.

4. The inhalator according to claim 1, wherein the fluid is a liquid.

5. The inhalator according to claim 4, wherein the fluid is water.

6. The inhalator according to claim 1, additionally comprising a heating/cooling means, wherein the temperature of the fluid is conditioned to a predetermined measure before being subjected to the respiratory tracts of the patient.

7. The inhalator according to claim 1, wherein the medicament is in solid phase.

8. The inhalator according to claim 6, wherein the medicament is selected from granular matter, fine particles, powder, sol, gel, sol-gel, glass, encapsulated matter, milled composition or any combination thereof.

9. The inhalator according to claim 1, wherein the medicament is in liquid phase.

10. The inhalator according to claim 8, wherein the medicament is a water miscible composition, water immiscible composition, emulsion, extract, dispersion, suspension, vesiculated solution, aggregated phase or any combination thereof.

11. The inhalator according to claim 1, wherein the medicament is being activated before or while being carried by the fluid column.

12. The inhalator according to claim 1, wherein the medicament is introduced to the dispenser by means of a disposable capsule comprising an envelope to be punctured or alternatively pierced such that a limited measure of the medicament contained in said enveloped is forced toward the pulsed column of fluid in a predetermined flux.

13. The inhalator according to claim 1, wherein the fluid oscillator is a rotating pistol.

14. The inhalator as defined in claim 1 or in any of its dependent claims, comprising:
a. air oscillator (103) providing a focused wind column with a series of alternating high and low pressures zones; and,
b. at least one outlet orifice (109) adapted to direct said focused fluid column towards or from the respiratory track of a patient,

15. The inhalator as defined in claim 1 or in any of its dependent claims, comprising:
a. a fluid oscillator (203) providing a focused fluid column with a series of alternating high and low pressures zones;
b. a medicament dispenser (209) adapted for releasing small and constant measures of at least one medicament;
c. a fluid dispenser (207); and,
d. at least one outlet orifice (212) adapted to direct said focused fluid column towards the respiratory track of a patient,
wherein said small and constant measures of medicament are subjected in said fluid to the patients while its respiratory tracks are gently and continuously vibrated.

16. The inhalator according to claim 1, wherein the fluid oscillator is a rotating disc.

17. The inhalator as defined in claim 1 or in any of its dependent claims, comprising:
a. a fluid turbine (307);
b. a medicament dispenser (304) adapted for releasing small and constant measures of at least one medicament via said fluid column;
c. a fluid and medicament oscillator (308) providing a focused wind or air column with a series of alternating high and low medicaments concentration; said oscillator is one or more rotating discs, especially a discs comprising a missing edge; and,
d. at least one outlet orifice (306) adapted to direct said focused fluid column towards the respiratory track of a patient,
wherein said small and constant measures of medicament are subjected to the patients while its respiratory tracks are gently and continuously vibrated.

18. The inhalator according to claim 1, additionally comprising at least one air flow valve adapted to provide medicament dispersing at both inhalation and exhalation steps of the respiratory mechanism.

19. The inhalator as defined in claim 1 or in any of its dependent claims, wherein the medicament delivery is provided by 2D or 3D specific and predetermined fitting mechanism or by any other physical parameter.

20. The inhalator according to claim 1 or to any of its dependent claims, additionally comprising one or more dispensing means being either active or passive means, adapted to force or purge at least a portion of the dispersed material towards a predetermined, e.g., focused target being either adjacent to the invented device or located in a remote location.

21. A method of inhaling small and constant measures of medicament while gently and continuously vibrating the patient's respiratory tracks by a means of a fluid column characterized by a series of alternating high and low pressures zones.

22. A method of treating upper respiratory diseases especially useful for reducing dyspnea, edema, spasm and collapse of the airways, comprising: vibrating the inhaled and/or exhaled air column while dispensing accurate and small dosage of medicaments to the respiratory tracks and peripheral regions wherein reducing the deposition of said medicament into the mouth cavity.

23. The method according to claim 21, especially adapted for preventing airways collapse during expiration by providing PCPAP.

24. The method according to claim 21, especially adapted for treating rhinitis and allergic rhinitis, rhinosinusitis, URTI, Asthma and COPD.
